(19) United States
(12) Patent Application Publication
(10) Pub. No.: US 2023/0338695 A1
(43) Pub. Date: Oct. 26, 2023

(54) CPAP APPARATUS

(71) Applicant: Murata Manufacturing Co., Ltd., Kyoto (JP)

(72) Inventors: Atsushi HATTORI, Kyoto (JP); Hsing KENGHUA, Kyoto (JP)

(21) Appl. No.: 18/302,852

(22) Filed: Apr. 19, 2023

(30) Foreign Application Priority Data
Apr. 20, 2022 (JP) 2022-069455

(51) Int. Cl. A61M 16/16; A61M 16/00; A61M 16/08

(52) U.S. Cl. CPC A61M 16/16; A61M 16/0066; A61M 16/0875

(57) ABSTRACT

A CPAP apparatus includes a blower and a humidifying tank having an inner space that is capable of storing water. The humidifying tank includes a bottom wall, a side wall, and a top wall. The humidifying tank includes a first passage through which the blower communicates with the inner space and a second passage through which the inner space communicates with a portion that is outside of the humidifying tank and that is different from the blower. When an inlet is defined as an opening of the first passage on the humidifying tank side, an outlet is defined as an opening of the second passage on the humidifying tank side, and an imaginary line segment is imagined to connect the inlet and the outlet along a shortest distance, the CPAP apparatus includes a shielding section that intersects the imaginary line segment.

### CROSS REFERENCE TO RELATED APPLICATION

This application claims priority from Japanese Patent Application No. 2022-069455 filed on Apr. 20, 2022. The content of this application is incorporated herein by reference in its entirety.

### BACKGROUND OF THE DISCLOSURE

### 1. Field of the Disclosure

The present disclosure relates to a CPAP apparatus.

### 2. Description of the Related Art

A CPAP apparatus described in Japanese Unexamined Patent Application Publication No. 2017-121502 includes a blower and a humidifying tank. The blower pressurizes and feeds air to the inside of the humidifying tank. The humidifying tank can store water in the inside thereof. The humidifying tank has a first passage and a second passage for allowing air to flow. The first passage connects the blower and the inside of the humidifying tank. The second passage connects the inside and the outside of the humidifying tank.

In the CPAP apparatus described in Japanese Unexamined Patent Application Publication No. 2017-121502, the air that has flowed into the humidifying tank from the blower via the first passage flows along the water surface of the water stored in the humidifying tank. Thus, the air is humidified. Here, the air may flow along the shortest distance from the first passage to the second passage in the humidifying tank. If the air flows along the shortest distance in this way, the flow speed of the air is considerably high even in the vicinity of the second passage. If the flow speed of the air is high in the vicinity of the second passage, the water surface may rise and fall to generate a wave in the vicinity of the second passage. Therefore, the wave at the water surface may wash over the second passage, or the water droplets formed by the wave may flow into the second passage.

### BRIEF SUMMARY OF THE DISCLOSURE

In order to solve the above problem, a CPAP apparatus according to the present disclosure includes: a blower that pressurizes and feeds air; and a humidifying tank that allows air, pressurized and fed by the blower, to flow thereinto and has an inner space that is capable of storing water. The humidifying tank includes: a top wall, a side wall, and a bottom wall that define the inner space; a first passage through which the blower communicates with the inner space; a second passage through which the inner space communicates with a portion that is outside of the humidifying tank and that is different from the blower; and a shielding section that intersects a line segment when an inlet is defined as an opening of the first passage on the humidifying tank side, an outlet is defined as an opening of the second passage on the humidifying tank side, and the line segment is imagined to connect the inlet and the outlet along a shortest distance. When a vertical axis is defined as a specific axis that intersects the bottom wall and a downward direction is defined as a direction that is along the vertical axis and that is from the top wall toward the bottom wall, an end of the shielding section in the downward direction is located on the downward direction side as seen from the inlet and on the downward direction side as seen from the outlet.

With the configuration described above, the shortest distance of the air flowing from the inlet toward the outlet is blocked by the shielding section. Accordingly, the route of the air is a route that circumvents the shielding section toward the outlet. By causing the air to flow along such a circumventing route, it is possible to reduce the flow speed of the air in the vicinity of the second passage. Thus, it is possible to prevent the water from flowing into the second passage due to the rising and falling of the water surface in the vicinity of the second passage.

The water in the humidifying tank is restrained from flowing into the outlet.

### BRIEF DESCRIPTION OF THE SEVERAL VIEWS OF THE DRAWINGS

FIG. 1 illustrates a state in which a CPAP apparatus is being used;

FIG. 2 is a perspective view of a humidifying tank;

FIG. 3 is a sectional view taken along line III-III in FIG. 4;

FIG. 4 is a sectional view taken along line IV-IV in FIG. 3; and

FIG. 5 is a sectional view taken along line V-V in FIG. 4.

### DETAILED DESCRIPTION OF THE DISCLOSURE

Hereafter, a CPAP apparatus according to an embodiment will be described. In the drawings, some elements may be enlarged for ease of understanding. The dimensional ratios of elements may differ from actual ones or from those in other figures.

### Overall Configuration

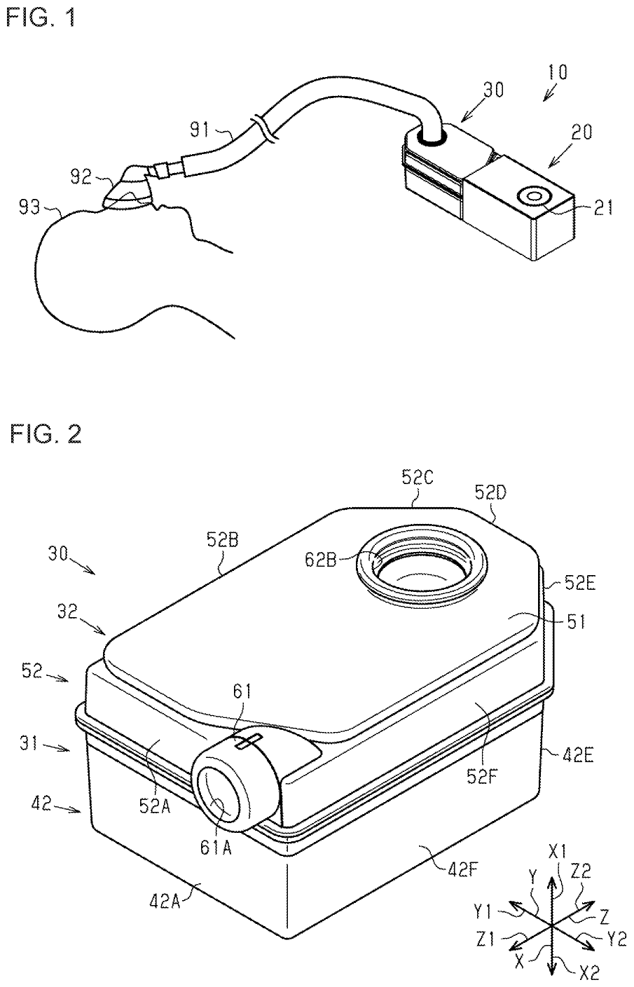

Referring to FIG. 1, the overall configuration of a continuous positive airway pressure apparatus (hereafter, referred to as a CPAP apparatus) will be described.

As illustrated in FIG. 1, a CPAP apparatus 10 includes a blower 20, a humidifying tank 30, a hose 91, and a mask 92.

The blower 20 is substantially shaped like a box. The blower 20 includes a blower fan for pressurizing and feeding air. The blower 20 has an air passage through which air flows between the inside and outside of the box. Illustration of the air passage is omitted in FIG. 1. The blower 20 has a switch 21. The switch 21 is located on an upper surface of the blower 20. The switch 21 is operated to control ON/OFF of the blower fan of the blower 20 and the like.

The humidifying tank 30 is connected to the blower 20. To be specific, the humidifying tank 30 is connected to the air passage of the blower 20. The humidifying tank 30 can store water in the inside thereof. The air that has been pressurized and fed from the blower fan of the blower 20 can flow to the inside of the humidifying tank 30.

The hose 91 is connected to the humidifying tank 30 at a position different from a position where the blower 20 is connected to the humidifying tank 30. The air that has passed through the inside of the humidifying tank 30 and has been humidified flows through the inside of the hose 91.

The mask 92 is connected to an end of the hose 91 opposite to an end on the humidifying tank 30 side. The mask 92 is worn by a user 93 so as to cover the nose or the mouth of the user 93. That is, the user 93 inhales the humidified air via the hose 91 and the mask 92.

### Humidifying Tank

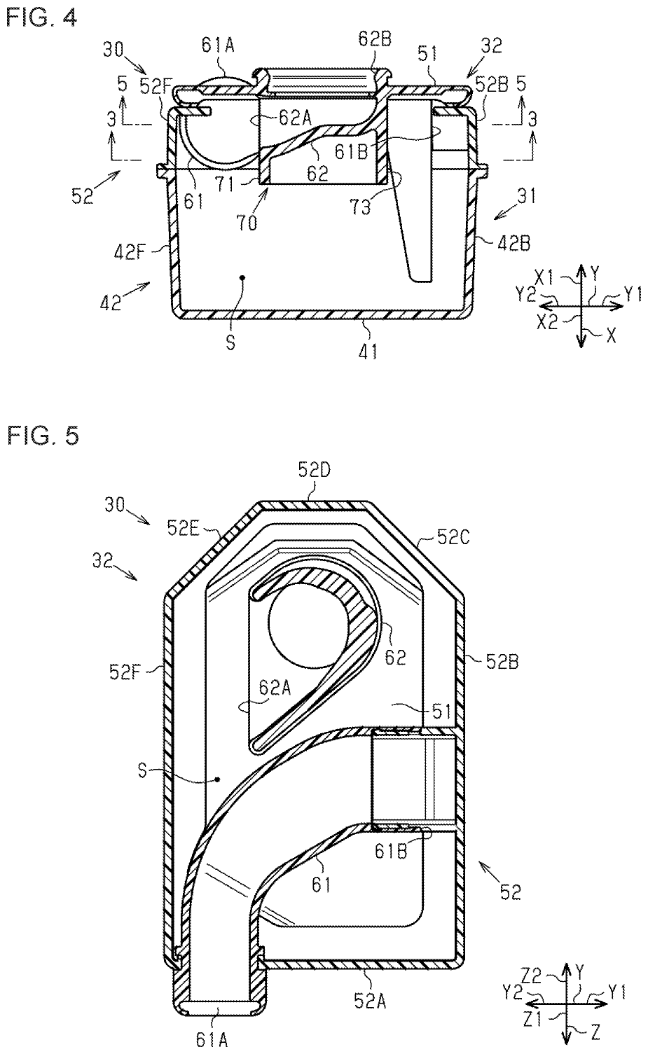

As illustrated in FIG. 2, the humidifying tank 30 has a tank portion 31 and a cover portion 32. As illustrated in FIG. 4, the tank portion 31 and the cover portion 32 define an inner space S of the humidifying tank 30.

As illustrated in FIG. 2, the tank portion 31 is shaped like a box that has a bottom and one side of which is open. That is, the tank portion 31 has a bottom wall 41 and a lower side wall 42 standing on the bottom wall 41.

The bottom wall 41 is shaped like a flat plate. The bottom wall 41 has a shape that is elongated in one direction when seen in a direction orthogonal to the bottom wall 41. To be specific, when seen in the direction orthogonal to the bottom wall 41, the bottom wall 41 has a shape such that two of the four corners of a rectangle are chamfered. Accordingly, the bottom wall 41 has a hexagonal shape having six sides when seen in the direction orthogonal to the bottom wall 41.

Here, a first axis X is defined as an axis orthogonal to the bottom wall 41. A second axis Y is defined as an axis orthogonal to the first axis X. In the present embodiment, the second axis Y is an axis extending in the transversal direction of the bottom wall 41. A third axis Z is defined as an axis orthogonal to the first axis X and the second axis Y. In the present embodiment, the third axis Z is an axis extending in the longitudinal direction of the bottom wall 41. A first positive direction X1 is defined as one of the two directions along the first axis X, and a first negative direction X2 is defined as the other direction opposite to the first positive direction X1. A second positive direction Y1 is defined as one of the two directions along the second axis Y, and a second negative direction Y2 is defined as the other direction opposite to the second positive direction Y1. A third positive direction Z1 is defined as one of the two directions along the third axis Z, and a third negative direction Z2 is defined as the other direction opposite to the third positive direction Z1. The humidifying tank 30 is used in a position such that the first positive direction X1 is the upward direction and the first negative direction X2 in the downward direction.

The lower side wall 42 stands in the first positive direction X1 on the edge of the bottom wall 41. The lower side wall 42 extends along the entire edge of the bottom wall 41. To be specific, the lower side wall 42 has a first lower side wall 42A, a second lower side wall 42B, a third lower side wall 42C, a fourth lower side wall (not shown), a fifth lower side wall 42E, and a sixth lower side wall 42F.

The first lower side wall 42A stands in the first positive direction X1 on an edge of the bottom wall 41 on the third positive direction Z1 side. The first lower side wall 42A is a wall orthogonal to the third axis Z.

The second lower side wall 42B stands in the first positive direction X1 on an edge of the bottom wall 41 on the second positive direction Y1 side. The second lower side wall 42B is a wall orthogonal to the second axis Y.

The third lower side wall 42C stands in the first positive direction X1 on an edge of the bottom wall 41 between the edge on the second positive direction Y1 side and an edge on the third negative direction Z2 side. That is, the third lower side wall 42C stands on an edge of a chamfered portion of the bottom wall 41. The third lower side wall 42C is a wall parallel to the first axis X. The third lower side wall 42C is inclined relative to both of the second axis Y and the third axis Z.

The fourth lower side wall (not shown) stands in the first positive direction X1 on the edge of the bottom wall 41 on the third negative direction Z2 side. The fourth lower side wall (not shown) is a wall orthogonal to the third axis Z. That is, the fourth lower side wall (not shown) is parallel to the first lower side wall 42A.

The fifth lower side wall 42E stands in the first positive direction X1 on an edge of the bottom wall 41 between the edge on the third negative direction Z2 side and an edge on the second negative direction Y2 side. That is, the fifth lower side wall 42E stands on an edge of a chamfered portion of the bottom wall 41. The fifth lower side wall 42E is a wall parallel to the first axis X. The fifth lower side wall 42E is inclined relative to both of the second axis Y and the third axis Z.

The sixth lower side wall 42F stands in the first positive direction X1 on the edge of the bottom wall 41 on the second negative direction Y2 side. The sixth lower side wall 42F is a wall orthogonal to the second axis Y. That is, the sixth lower side wall 42F is parallel to the second lower side wall 42B.

The cover portion 32 has an upper cover 51 and an upper side wall 52. The cover portion 32 covers the opening of the tank portion 31 on the first positive direction X1 side.

When seen in a direction along the first axis X, the outer shape of the upper cover 51 is the same as that of the bottom wall 41. That is, the bottom wall 41 has a shape such that two corners of a rectangle are chamfered. The upper cover 51 and the bottom wall 41 face each other in the direction along the first axis X. In the present embodiment, the first axis X, which is orthogonal to the bottom wall 41 and the upper cover 51, is the vertical axis. A direction from the upper cover 51 toward the bottom wall 41 is the downward direction. A direction from the bottom wall 41 toward the upper cover 51 is the upward direction.

The upper side wall 52 stands in the first negative direction X2 on the edge of the upper cover 51. The upper side wall 52 extends along the entire edge of the upper cover 51. To be specific, the upper side wall 52 has a first upper side wall 52A, a second upper side wall 52B, a third upper side wall 52C, a fourth upper side wall 52D, a fifth upper side wall 52E, and a sixth upper side wall 52F.

The first upper side wall 52A stands in the first negative direction X2 on an edge of the upper cover 51 on the third positive direction Z1 side. The first upper side wall 52A is a wall orthogonal to the third axis Z.

The second upper side wall 52B stands in the first negative direction X2 on an edge of the upper cover 51 on the second positive direction Y1 side. The second upper side wall 52B is a wall orthogonal to the second axis Y.

The third upper side wall 52C stands in the first negative direction X2 on an edge of the upper cover 51 between the edge on the second positive direction Y1 side and an edge on the third negative direction Z2 side. That is, the third upper side wall 52C stands on an edge of a chamfered portion of the upper cover 51. The third upper side wall 52C is a wall parallel to the first axis X. The third upper side wall 52C is inclined relative to both of the second axis Y and the third axis Z.

The fourth upper side wall 52D stands in the first negative direction X2 on the edge of the upper cover 51 on the third negative direction Z2 side. The fourth upper side wall 52D is a wall orthogonal to the third axis Z. That is, the fourth upper side wall 52D is parallel to the first upper side wall 52A.

The fifth upper side wall 52E stands in the first negative direction X2 on an edge of the upper cover 51 between the edge on the third negative direction Z2 side and an edge on the second negative direction Y2 side. That is, the fifth upper side wall 52E stands on an edge of a chamfered portion of the upper cover 51.

The sixth upper side wall 52F stands in the first negative direction X2 on the edge of the upper cover 51 on the second negative direction Y2 side. The sixth upper side wall 52F is a wall orthogonal to the second axis Y. That is, the sixth upper side wall 52F is parallel to the second upper side wall 52B.

An end of the upper side wall 52 on the first negative direction X2 side is connected with an end of the lower side wall 42 on the first positive direction X1 side. To be specific, the first upper side wall 52A is connected with the first lower side wall 42A. The second upper side wall 52B is connected with the second lower side wall 42B. The third upper side wall 52C is connected with the third lower side wall 42C. The fourth upper side wall 52D is connected with the fourth lower side wall (not shown). The fifth upper side wall 52E is connected with the fifth lower side wall 42E. The sixth upper side wall 52F is connected with the sixth lower side wall 42F. The upper side wall 52 and the lower side wall 42 constitute the side wall of the humidifying tank 30.

### Passages

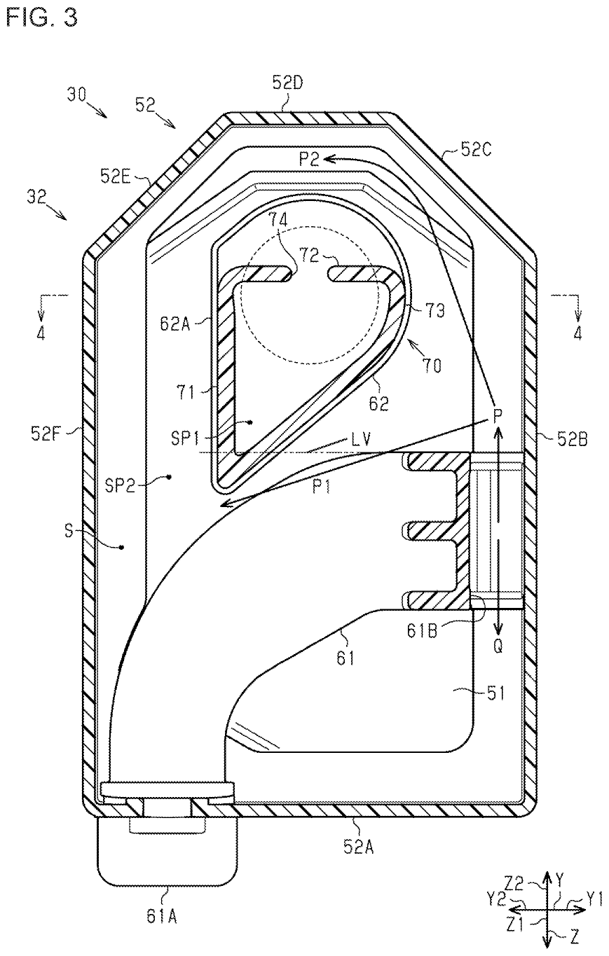

As illustrated in FIG. 3, the cover portion 32 has a first passage 61 and a second passage 62.

The first passage 61 has a tubular shape. The first passage 61 extends through the first upper side wall 52A. That is, the first passage 61 is a path through which the blower 20 communicates with the inner space S of the humidifying tank 30. The first passage 61 is made of a material different from that of the cover portion 32.

As illustrated in FIG. 3, a first end 61A of the first passage 61 is located outside of a space defined by the first to sixth upper side walls 52A to 52F. The shape of an opening in the first end 61A of the first passage 61 is substantially circular. The opening in the first end 61A of the first passage 61 faces in the third positive direction Z1. The first end 61A of the first passage 61 is connected to the blower 20.

The direction in which an opening faces is determined as follows. First, a viewpoint from which the apparent opening area of the opening is the maximum as seen from the outside is determined. Then, the direction in which the opening faces is defined as a direction toward the viewpoint from the geometric center of the opening as seen from the viewpoint.

As illustrated in FIG. 4, a second end 61B of the first passage 61 is located in the inner space S of the humidifying tank 30. The shape of the opening at the second end 61B of the first passage 61 is substantially quadrangular. The opening at the second end 61B of the first passage 61 faces in the second positive direction Y1. That is, as illustrated in FIG. 3, the first passage 61 is curved by 90 degrees from the first end 61A toward the second end 61B. The opening at the second end 61B of the first passage 61 faces the second upper side wall 52B. The opening at the second end 61B of the first passage 61, that is, the opening on the inner space S side is an inlet of air.

As illustrated in FIG. 4, the second passage 62 has a tubular shape. The second passage 62 extends through the upper cover 51. That is, the second passage 62 is a path through which the inner space S of the humidifying tank 30 communicates with a region that is outside of the humidifying tank 30 and different from the blower 20.

The second passage 62 is integrally formed with the upper cover 51. A part of the outer surface of the second passage 62 faces the bottom wall 41 in a direction along the second axis Y. That is, the second passage 62 and the upper cover 51 constitute a top wall of the humidifying tank 30.

A first end 62A of the second passage 62 is located in the inner space S of the humidifying tank 30. The shape of an opening at the first end 62A of the second passage 62 is substantially quadrangular. The opening at the first end 62A of the second passage 62 faces in the second negative direction Y2. The opening at the first end 62A of the second passage 62, that is, the opening of the second passage 62 on the humidifying tank 30 side is an outlet of air.

A second end 62B of the second passage 62 is located outside of the humidifying tank 30. The shape of an opening at the second end 62B of the second passage 62 is substantially circular. The opening at the second end 62B of the second passage 62 faces in the first positive direction X1. That is, the second passage 62 is curved by 90 degrees from the first end 62A toward the second end 62B. Accordingly, the second passage 62 extends so as to be located gradually in the first positive direction X1 with increasing distance from the first end 62A toward the second end 62B. The second end 62B of the second passage 62 is connected to the hose 91.

As illustrated in FIG. 5, an outer surface of the second passage 62 on the third negative direction Z2 side has an arc-like shape. The shortest distance from the outer surface of the second passage 62 to the third upper side wall 52C, the shortest distance from the outer surface of the second passage 62 to the fourth upper side wall 52D, and the shortest distance from the outer surface of the second passage 62 to the fifth upper side wall 52E are substantially the same as each other.

### Shielding Section

As illustrated in FIG. 4, the humidifying tank 30 has a shielding section 70. The shielding section 70 has a tubular shape extending along the first axis X. The shielding section 70 extends in the first negative direction X2 from the outer surface of the second passage 62. As described above, the second passage 62 is a part of the top wall of the humidifying tank 30. Accordingly, the shielding section 70 extends from the top wall toward the first negative direction X2 side. The shielding section 70 divides the inner space S into a first space SP1 surrounded by the shielding section 70 and a second space SP2 opposite to the first space SP1 with the shielding section 70 therebetween. In other words, the first space SP1 is the inner space of the tube of the shielding section 70. The second space SP2 is the outer space of the tube of the shielding section 70.

Here, as illustrated in FIG. 3, an imaginary line segment LV is imagined to connect the second end 61B of the first passage 61 and the first end 62A of the second passage 62 along the shortest distance. The shielding section 70 intersects the imaginary line segment LV.

As illustrated in FIG. 4, an end of the shielding section 70 on the first negative direction X2 side is located further toward the first negative direction X2 side than the connection position where the upper side wall 52 and the lower side wall 42 are connected. That is, the end of the shielding section 70 on the first negative direction X2 side is located on the first negative direction X2 side as seen from the second end 61B of the first passage 61. The end of the shielding section 70 on the first negative direction X2 side is located on the first negative direction X2 side as seen from the first end 62A of the second passage 62. When water is stored in the humidifying tank 30 to a full level that is supposed during the use of the CPAP apparatus 10, a part of the shielding section 70 including the end on the first negative direction X2 side is immersed in water.

As illustrated in FIG. 3, the shielding section 70 has a substantially triangular tubular shape. To be specific, the shielding section 70 has a first flat plate 71, a second flat plate 72, and a third flat plate 73. The first flat plate 71 extends from the first end 62A of the second passage 62 toward the first negative direction X2 side. The first flat plate 71 is a flat plate that is orthogonal to second axis Y.

The second flat plate 72 extends from the lower surface of the second passage 62 in the first negative direction X2. The second flat plate 72 is a flat plate that is orthogonal to the third axis Z. The second flat plate 72 is connected to an end of the first flat plate 71 on the third negative direction Z2 side.

The third flat plate 73 extends from the lower surface of the second passage 62 in the first negative direction X2. The third flat plate 73 is a flat plate that is parallel to the first axis X. The third flat plate 73 is inclined relative to both of the second axis Y and the third axis Z. An end of the third flat plate 73 on the third negative direction Z2 side is connected to an end of the second flat plate 72 on the second positive direction Y1 side. An end of the third flat plate 73 on the third positive direction Z1 side is connected to an end of the first flat plate 71 on the third positive direction Z1 side. A portion of the third flat plate 73 in the vicinity of a connection portion where the third flat plate 73 is connected with the second flat plate 72 is curved so as to be convex toward the second positive direction Y1 side.

When seen in the direction along the first axis X, in the shielding section 70, the first flat plate 71 and the second flat plate 72 do not face each other parallelly. Likewise, the first flat plate 71 and the third flat plate 73 do not face each other parallelly. Moreover, the second flat plate 72 and the third flat plate 73 do not face each other parallelly. That is, the inner surface of the shielding section 70 does not have portions that face each other parallelly.

The shielding section 70 has a through-hole 74. The through-hole 74 is located at the center of the second flat plate 72 in the direction along the second axis Y. Through the through-hole 74, the first space SP1 communicates with the second space SP2. The shape of the through-hole 74 is a groove-like shape extending along the first axis X. The through-hole 74 extends along the entire length of the second flat plate 72 in the direction along the first axis X. As described above, the end of the shielding section 70 on the first negative direction X2 side is located on the first negative direction X2 side as seen from the second end 61B of the first passage 61. Accordingly, the end of the through-hole 74 on the first negative direction X2 side is located on the first negative direction X2 side as seen from the second end 61B of the first passage 61. Likewise, the end of the through-hole 74 on the first negative direction X2 side is located on the first negative direction X2 side as seen from the first end 62A of the second passage 62.

When seen in the direction along the first axis X, the through-hole 74 faces in the third negative direction Z2. Accordingly, the through-hole 74 faces in a direction different from the direction in which the opening of the first passage 61 on the second end 61B side faces. The third flat plate 73 is a facing surface that faces the through-hole 74 in the direction along the third axis Z in which the through-hole 74 faces. When seen in the direction along the first axis X, the third flat plate 73 is inclined relative to the opening surface of the through-hole 74.

### Operations of Present Embodiment

When using the CPAP apparatus 10, the CPAP apparatus 10 is placed on a desk or the like so that the first positive direction X1 of the CPAP apparatus 10 coincides with the upward direction and the first negative direction X2 of the CPAP apparatus 10 coincides with the downward direction. When using the CPAP apparatus 10, water is stored in the inner space S of the humidifying tank 30. At this time, water is supplied in an amount with which the end of the shielding section 70 on the first negative direction X2 side is immersed in the water.

When the switch 21 is pressed, the blower 20 is driven, and the compressed air is fed from the blower fan. The air passes through the first passage 61 of the humidifying tank 30, and flows into the humidifying tank 30.

The air that has flowed into the humidifying tank 30 flows in a space that is included in the inner space S of the humidifying tank 30 and that is on the first positive direction X1 side as seen from the water surface. At this time, as illustrated in FIG. 3, the flow of the air that has entered from the inlet at the second end 61B of the first passage 61 collides with the second lower side wall 42B and the second upper side wall 52B. Subsequently, the flow of the air is divided into a route P toward the third negative direction Z2 side and a route Q toward the third positive direction Z1 side.

As described above, the shielding section 70 is present on the imaginary line segment LV that shows the shortest distance from the inlet to the outlet. Accordingly, the air that has been discharged toward the route P does not pass along the shortest route and flows so as to circumvent the shielding section 70. The circumventing route includes two major routes. The two routes include a route P1 on the third positive direction Z1 side as seen from the shielding section 70 and a route P2 between the lower and upper side walls 42 and 52 of the humidifying tank 30 and the shielding section 70.

Among the routes Q, P1, and P2, the route Q has the largest path sectional area. At a portion where the path sectional area is large, the flow speed of the air is low, the pressure of the air is comparatively high, and thus the air does not easily flow into the portion. Accordingly, a larger amount of the air flows into the route P1 and the route P2 than into the route Q. The path sectional area of the route P2 does not change considerably. Thus, the air flowing along the route P2 does not cause turbulent flow or the like that is not intended in design.

### Effects of Present Embodiment

(1) With the embodiment described above, the route of the air is a route that circumvents the shielding section 70 toward the first end 62A of the second passage 62. By causing the air to flow along the circumventing route, it is possible to reduce the flow speed of the air in the vicinity of the second passage 62. Thus, it is possible to prevent the water from flowing into the second passage 62 due to the rising and falling of the water surface in the vicinity of the second passage 62.

(2) With the embodiment described above, the end of the shielding section 70 on the first negative direction X2 side is located on the first negative direction X2 side as seen from the second end 61B of the first passage 61 and on the first negative direction X2 side as seen from the first end 62A of the second passage 62. With such a configuration, it is possible to supply the water into the humidifying tank 30 so that the lower end of the shielding section 70 is immersed. In such a state, the air cannot pass through the space between the shielding section 70 and the water surface. That is, as the route of the air, it is possible to form a route that reliably circumvents the shielding section 70.

(3) With the embodiment described above, when the water surface in the humidifying tank 30 is located on an upper side as seen from the lower end of the through-hole 74, the water is present also in the first space SP1. In this state, the waves at the water surface generated in the humidifying tank 30 propagate from the second space SP2 to the first space SP1. The waves propagated into the first space SP1 interfere with each other and cancel each other out in the first space SP1. That is, with the configuration described above, the shielding section 70 can perform a function of attenuating the waves at the water surface in the humidifying tank 30.

(4) With the embodiment described above, the air that has flowed into the humidifying tank 30 from the inlet at the second end 61B of the first passage 61 collides with the second lower side wall 42B and the second upper side wall 52B and flows toward the bottom wall 41 of the humidifying tank 30. At this time, the water surface between the inlet at the second end 61B of the first passage 61 and the second lower and upper side walls 42B and 52B is lowered toward the bottom wall 41. Due to such a movement of the water surface, in the humidifying tank 30, a wave is generated to propagate in a direction that intersects the second axis Y along which the inlet at the second end 61B of the first passage 61 faces. With the configuration described above, the through-hole 74 of the shielding section 70 faces in a direction different from the direction in which the inlet at the second end 61B of the first passage 61 faces. Therefore, the wave that propagates in the direction that intersects the second axis Y easily passes through the through-hole 74 and propagates to the first space SP1.

(5) With the embodiment described above, a wave that has propagated to the first space SP1 via the through-hole 74 collides with and reflected by the third flat plate 73. At this time, the reflected wave propagates in a direction different from the direction in which the through-hole 74 is present. Accordingly, it is possible to prevent the reflected wave from propagating to the outside of the shielding section 70 via the through-hole 74.

(6) In the embodiment described above, the inner surface of the shielding section 70 does not have portions that face each other parallelly. Accordingly, a wave that has propagated to the first space SP1 via the through-hole 74 collides with the inner surface of the shielding section 70 and is reflected in various directions. Therefore, the reflected waves easily interfere with each other. As a result, the reflected waves are efficiently attenuated.

(7) In the embodiment described above, the second passage 62 extends so as to be located gradually in the first positive direction X1 with increasing distance from the outlet, which is the opening on the first end 62A side, toward the downstream side. With this configuration, even if water enters the second passage 62, the water flows to the inside of the humidifying tank 30 due to gravity.

(8) In the embodiment described above, the pass sectional area of the route P2 does not change considerably. Accordingly, the air flowing along the route P2 does not cause turbulent flow or the like that is not intended in design.

### Modifications

The present embodiment and modifications described below can be used in combination as long as technological contradiction does not occur.

In the embodiment described above, the CPAP apparatus 10 may have any configuration provided that the CPAP apparatus has the blower 20 and the humidifying tank 30. The shape of the entirety of the CPAP apparatus 10 is not limited.

In the embodiment described above, the configuration of the humidifying tank 30 is not limited. For example, the tank portion 31 and the cover portion 32 may be integrally formed, and a part of the side wall of the tank portion 31 may be open.

In the embodiment described above, the humidifying tank 30 may be quadrangular or may be circular when seen in the direction along the first axis X.

In the embodiment described above, the first passage 61 may be integrally formed with the cover portion 32.

In the embodiment described above, the shape of the first passage 61 is not limited, as long as air can flow through the inside thereof. For example, the cross section of the first passage 61 may be uniform from the first end 61A to the second end 61B. A part of the path of the first passage 61 may be formed by the upper cover 51. The first passage 61 may extend linearly.

In the embodiment described above, the shape of the second passage 62 is not limited, as long as air can flow through the inside thereof. For example, the path cross section the second passage 62 may be uniform from the first end 62A to the second end 62B. The second passage 62 may extend linearly and parallel to the upper cover 51. That is, the second end 62B of the second passage 62 may protrude from the side wall of the tank portion 31. The second passage 62 may be curved toward the first negative direction X2 from the first end 62A of the second passage 62 toward the downstream side.

In the embodiment described above, the end of the shielding section 70 on the first negative direction X2 side may reach the bottom wall 41.

In the embodiment described above, the shape of the shielding section 70 is not limited to the example in the embodiment described above. For example, when seen in the direction along the first axis X, the shielding section 70 may have a circular shape or a quadrangular shape. The facing surface of the inner surface of the shielding section 70 facing the through-hole 74 may be parallel to the direction in which the through-hole 74 faces.

In the embodiment described above, the shielding section 70 may extend toward the first negative direction X2 side from the upper cover 51. As long as the shielding section 70 extends from the top wall that faces the bottom wall 41, air does not flow through the gap between the shielding section 70 and the top wall.

In the embodiment described above, the shielding section 70 need not have a tubular shape. For example, the shielding section 70 may have a plate-like shape. Also in the case where the shielding section 70 has a plate-like shape, it is sufficient that the shielding section 70 is disposed so as to intersect the imaginary line segment LV.

In the embodiment described above, the position of the through-hole 74 relative to the shielding section 70 is not limited to the example in the embodiment described above. For example, the through-hole 74 may face in a direction that is the same as the direction in which the opening of the first passage 61 on the second end 61B side faces.

In the embodiment described above, the through-hole 74 need not reach an end of the shielding section 70 on the first negative direction X2 side in the direction along the first axis X. It is sufficient that, when the CPAP apparatus 10 is being used, the humidifying tank 30 can store water so that the water surface can be located on the first positive direction X1 side relative to the end of the through-hole 74 on the first negative direction X2 side.

In the embodiment described above, the shape of the opening of the through-hole 74 is not limited. For example, the through-hole 74 may have a circular shape or an elliptical shape when seen from the direction in which the through-hole 74 faces.

Technological ideas that can be grasped from the embodiment and the modifications described above will be described.

[1] A CPAP apparatus comprising: a blower that pressurizes and feeds air; and a humidifying tank that allows air, pressurized and fed by the blower, to flow thereinto and has an inner space that is capable of storing water, wherein the humidifying tank includes a top wall, a side wall, and a bottom wall that define the inner space, a first passage through which the blower communicates with the inner space, a second passage through which the inner space communicates with a portion that is outside of the humidifying tank and that is different from the blower, and a shielding section that intersects a line segment when an inlet is defined as an opening of the first passage on the humidifying tank side, an outlet is defined as an opening of the second passage on the humidifying tank side, and the line segment is imagined to connect the inlet and the outlet along a shortest distance, and wherein, when a vertical axis is defined as a specific axis that intersects the bottom wall and a downward direction is defined as a direction that is along the vertical axis and that is from the top wall toward the bottom wall, an end of the shielding section in the downward direction is located on the downward direction side as seen from the inlet and on the downward direction side as seen from the outlet.

[2] The CPAP apparatus described in [1], wherein the shielding section has a tubular shape, wherein the shielding section has a through-hole through which a first space surrounded by the shielding section communicates with a second space opposite to the first space with the shielding section therebetween, and wherein a lower end of the through-hole is located on the downward direction side as seen from the inlet and on the downward direction side as seen from the outlet.

[3] The CPAP apparatus described in [2], wherein the inlet faces the side wall, and wherein, when seen in a direction along the vertical axis, the through-hole faces in a direction different from a direction in which the inlet faces.

[4] The CPAP apparatus described in [2] or [3], wherein an inner surface of the shielding section includes a facing surface that faces the through-hole in a direction in which the through-hole faces, and wherein, when seen in a direction along the vertical axis, the facing surface is inclined relative to an opening surface of the through-hole.

[5] The CPAP apparatus described in any one of [2] to [4], wherein, when seen in a direction along the vertical axis, an inner surface of the shielding section does not have portions that face each other parallelly.

[6] The CPAP apparatus described in any one of [1] to [5], wherein, when an upward direction is defined as a direction that is along the vertical axis and that is toward the second passage as seen from the bottom wall, the second passage extends so as to be located in the upward direction with increasing distance from the outlet toward a downstream side.

What is claimed is:

1. A continuous positive airway pressure apparatus comprising:
a blower configured to pressurize and feed air; and
a humidifying tank configured to allow the air, pressurized and fed by the blower, to flow thereinto, the humidifying tank having an inner space capable of storing water,
wherein the humidifying tank includes
a top wall, a side wall, and a bottom wall defining the inner space,
a first passage through which the blower communicates with the inner space,
a second passage through which the inner space communicates with a portion being outside of the humidifying tank and being different from the blower, and
a shielding section intersecting a line segment when an inlet is defined as an opening of the first passage on the humidifying tank side, an outlet is defined as an opening of the second passage on the humidifying tank side, and the line segment is imagined to connect the inlet and the outlet along a shortest distance, and
wherein, when a vertical axis is defined as a specific axis intersecting the bottom wall and a downward direction is defined as a direction being along the vertical axis and being from the top wall toward the bottom wall,
an end of the shielding section in the downward direction is located toward the downward direction as seen from the inlet and toward the downward direction as seen from the outlet.

2. The continuous positive airway pressure apparatus according to claim 1,
wherein the shielding section has a tubular shape,
wherein the shielding section has a through-hole through which a first space surrounded by the shielding section communicates with a second space opposite to the first space with the shielding section therebetween, and
wherein a lower end of the through-hole is located toward the downward direction as seen from the inlet and toward the downward direction as seen from the outlet.

3. The continuous positive airway pressure apparatus according to claim 2,
wherein the inlet faces the side wall, and
wherein, when seen in a direction along the vertical axis, the through-hole faces in a direction different from a direction in which the inlet faces.

4. The continuous positive airway pressure apparatus according to claim 2,
wherein an inner surface of the shielding section includes a facing surface facing the through-hole in a direction in which the through-hole faces, and
wherein, when seen in a direction along the vertical axis, the facing surface is inclined relative to an opening surface of the through-hole.

5. The continuous positive airway pressure apparatus according to claim 2,
wherein, when seen in a direction along the vertical axis, an inner surface of the shielding section does not have portions facing each other parallelly.

6. The continuous positive airway pressure apparatus according to claim 1,
wherein, when an upward direction is defined as a direction being along the vertical axis and being toward the second passage as seen from the bottom wall,
the second passage extends so as to be located in the upward direction with increasing distance from the outlet toward a downstream side.

7. The continuous positive airway pressure apparatus according to claim 3,
wherein an inner surface of the shielding section includes a facing surface facing the through-hole in a direction in which the through-hole faces, and
wherein, when seen in a direction along the vertical axis, the facing surface is inclined relative to an opening surface of the through-hole.
